(19) United States
(12) Patent Application Publication
(10) Pub. No.: US 2026/0020926 A1
(43) Pub. Date: Jan. 22, 2026

(54) INSTRUMENT FOLLOW ORIENTATION OFFSET FOR ERGONOMIC SURGEON MANIPULATOR CONTROL ORIENTATION

(71) Applicant: Intuitive Surgical Operations, Inc., Sunnyvale, CA (US)

(72) Inventor: Arpit Mittal, Redwood City, CA (US)

(21) Appl. No.: 19/270,089

(22) Filed: Jul. 15, 2025

Related U.S. Application Data
(60) Provisional application No. 63/673,377, filed on Jul. 19, 2024.

(51) Int. Cl. A61B 34/35; A61B 34/00; A61B 34/30; B25J 9/16; B25J 13/06

(52) U.S. Cl. CPC A61B 34/35; A61B 34/25; B25J 9/1689; B25J 13/065; A61B 2034/305

(57) ABSTRACT

Systems and methods are described for assisting a user in controlling an instrument. The method may include: while operating according to a first operating condition: configuring a display device to display an actual representation of the instrument shaft based on image data; and controlling motion of the instrument working portion and shaft; and while operating according to a second operating condition: modifying image data to include a virtual representation of the instrument shaft and obscure the actual representation; configuring the display device to display the modified image data; and controlling motion of the instrument working portion and shaft by: receiving control signals from the user input system, processing control signals to determine a desired motion relative to the actual position to generate one or more modified control signals, and causing the repositionable structure to move relative to the actual position of the instrument shaft based on the modified control signals.

### CROSS-REFERENCE TO RELATED APPLICATIONS

This application claims priority to and the benefit of the filing date of provisional U.S. Patent Application No. 63/673,377 entitled “INSTRUMENT FOLLOW ORIENTATION OFFSET FOR ERGONOMIC SURGEON MANIPULATOR CONTROL ORIENTATION,” filed on Jul. 19, 2024. The entire contents of the provisional application are hereby expressly incorporated herein by reference.

### FIELD

Disclosed examples relate to guided robotic control manipulator systems. In particular, the disclosed examples relate to systems and methods for controlling operation of an instrument supported by a repositionable structure and displaying an actual or virtual representation of the instrument to a user.

### BACKGROUND

Computer-assisted manipulator systems (“manipulator systems”), sometimes referred to as robotically assisted systems or robotic systems, may include one or more manipulators that can be operated and/or teleoperated with the assistance of a user input device to move and control functions of one or more instruments coupled to the manipulators. A manipulator generally includes mechanical links connected by joints. An instrument is removably (or permanently) coupled to one of the links, typically a distal link of the plural links.

In some computer-assisted manipulator systems, the manipulators are attached to a manipulator support structure (e.g., a patient side cart) that is separate from a support structure that supports a patient or workpiece. In other manipulator systems, the manipulators are attached directly to the support structure (herein referred to as a “table assembly”) that supports the patient or workpiece (e.g., to an operating table). Manipulator systems in which the manipulators are mounted to the table assembly can be referred to herein as table-mounted manipulator systems.

An important aspect of utilizing computer-assisted manipulator systems, is the ability for a user to ergonomically and comfortably use the manipulator system. In particular, a user performing an operation on a target should be able to carry out the procedure using the user input device from an ergonomically neutral and/or comfortable position to ensure maximum stability and capability in performing the operation. However, as instruments for performing such operations improve in overall capability, the human wrist becomes an increasingly limiting factor in manipulating the user input device to control positioning of the instrument. For example, in performing a complex procedure with a manipulator system, the placement of a port (e.g., entry point, insertion point, remote center, etc.) may be in difficult to reach spots. Conventional systems may result in manual manipulation of the user input devices at a strange or difficult angle when attempting to perform these complex procedures.

Accordingly, a need exists for improved manipulator systems with an ability to determine and display to a user an improved approach to a port, including a direction and angle of approach.

### SUMMARY

The following presents a simplified summary of various examples described herein and is not intended to identify key or critical elements or to delineate the scope of the claims.

In some examples, a computer-assisted system for assisting a user in controlling an instrument including an instrument working portion coupled to an instrument shaft via an instrument wrist is provided. The computer-assisted system may comprise: a repositionable structure configured to support (i) the instrument working portion via the instrument shaft and (ii) an image sensor configured to generate image data representative of the instrument working portion and the instrument shaft; a user input system; an output system comprising a display device; and a control system operably coupled to the repositionable structure, the user input system, and the output system, the control system configured to: (a) while operating according to a first operating condition, (i) configure the display device to display an actual representation of the instrument shaft based on the image data and (ii) control motion of the instrument working portion and the instrument shaft by: (1) receiving one or more first control signals for controlling the repositionable structure from the user input system, and (2) causing the repositionable structure to move relative to an actual position of the instrument shaft based on the one or more first control signals; (b) while operating according to a second operating condition, modify the image data to include a virtual representation of the instrument shaft and obscure the actual representation of the instrument shaft; and (c) while operating according to the second operating condition, (i) configure the display device to display the modified image data and (ii) control motion of the instrument working portion and the instrument shaft by: (1) receiving one or more second control signals for controlling the repositionable structure from the user input system, (2) processing the one or more second control signals to determine a desired motion relative to the actual position to generate one or more modified second control signals, and (3) causing the repositionable structure to move relative to the actual position of the instrument shaft based on the one or more modified second control signals.

In further examples, a computer-implemented method for assisting a user in controlling an instrument including an instrument working portion coupled to an instrument shaft via an instrument wrist is provided. The computer-implemented method may comprise: while operating according to a first operating condition: (a) configuring, by one or more processors of a control system operably coupled to a repositionable structure configured to support (i) the instrument working portion via the instrument shaft and (ii) an image sensor configured to generate image data representative of the instrument working portion and the instrument shaft, a display device of an output system to display an actual representation of the instrument shaft based on the image data; and (b) controlling, by the one or more processors, motion of the instrument working portion and the instrument shaft by: (1) receiving, by the one or more processors, one or more first control signals for controlling the repositionable structure from a user input system, and (2) causing, by the one or more processors, the repositionable structure to move relative to an actual position of the instrument shaft based on the one or more first control signals; and while operating according to a second operating condition: (a) modifying, by the one or more processors, the image data to include a virtual representation of the instrument shaft and obscure the actual representation of the instrument shaft; (b) configuring, by the one or more processors, the display device to display the modified image data; and (c) controlling, by the one or more processors, motion of the instrument working portion and the instrument shaft by: (1) receiving, by the one or more processors, one or more second control signals based on the virtual representation of the instrument and for controlling the repositionable structure from the user input system, (2) processing, by the one or more processors, the one or more second control signals to determine a desired motion relative to the actual position to generate one or more modified second control signals, and (3) causing, by the one or more processors, the repositionable structure to move relative to the actual position of the instrument shaft based on the one or more modified second control signals.

In still further examples, a non-tangible computer-readable medium storing instructions for assisting a user in controlling an instrument is provided. The instructions, when executed, may cause a control system operably coupled to a repositionable structure to perform the methods described herein.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a schematic diagram for a robotically-assisted manipulator system for controlling operation of an instrument supported by a repositionable structure and displaying an actual or virtual representation of the instrument to a user according to some examples.

FIG. 2 is a front view of an embodiment of a console for controlling the manipulator system of FIG. 1 and displaying an actual or virtual representation of the instrument to a user.

FIG. 3 is a block diagram of a system including multiple manipulators, control devices, and outputs for displaying an actual or virtual representation of the instrument to a user.

FIG. 4A is a diagrammatic illustration of an endoscopic view including an actual representation of an instrument and a positioning for controllers in accordance with the endoscopic view, according to some examples.

FIG. 4B is a diagrammatic illustration of an endoscopic view including a virtual representation of an instrument and a positioning for controllers in accordance with the endoscopic view, according to some examples.

FIG. 5 is an example flow diagram of an example method for controlling operation of an instrument supported by a repositionable structure and displaying an actual or virtual representation of the instrument to a user, according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In the following description, specific details are set forth describing some examples consistent with the present disclosure. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, components, and circuits have not been described in detail so as not to unnecessarily obscure aspects of the examples.

This disclosure describes various instruments and portions of instruments in terms of their state in three-dimensional space. The instruments may be any sort of instrument used to perform a procedure as described herein (e.g., a flexible instrument, a semi-rigid instrument, a rigid instrument, etc.). As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

This disclosure occasionally refers to the disclosed techniques being applied to “patients” undergoing a “medical procedure.” It should be appreciated that these references are not intended to limit the application of the disclosed techniques to applied medicine contexts. For example, the described techniques can be applied to facilitate physician training, equipment testing and/or calibration, and/or other contexts. Accordingly, any reference to the term “patient” is done for case of explanation and also envisions the application of the described techniques to a generic “subject” or “target.”

Aspects of this disclosure herein can be part of a computer-assisted manipulator system, sometimes referred to as a robotically-assisted manipulator system or a robotic system. The manipulator system can include one or more manipulators that can be operated manually and/or with the assistance of an electronic controller (e.g., computer) to move and control functions of one or more instruments when coupled to the manipulators. Embodiments where a manipulator system is electronically controlled, at least in part, may be referred to as a teleoperational manipulator system.

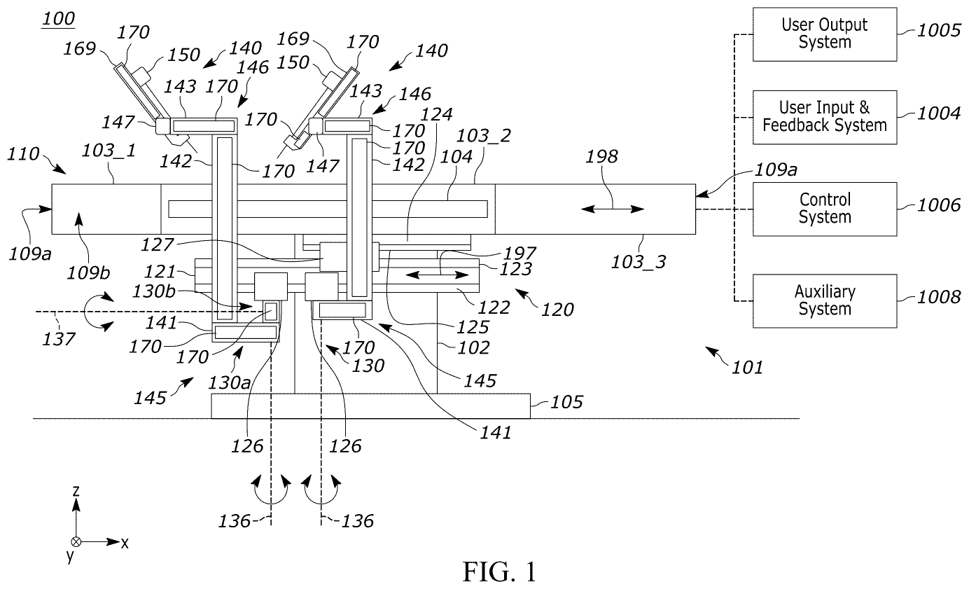

FIG. 1 illustrates an embodiment of a table-mounted manipulator system 100 (“system 100”) for performing procedures via a control system 1006 that operates one or more components of the system 100 and provides a view of such to a user. The view provided to the user may be representative of an actual pose (e.g., location, position, angle, etc.) of an instrument and/or of a virtual representation of such, in which the virtual representation depicts a same endpoint, but allows a user to reposition controls based on one or more metrics (e.g., for comfort, for vision, etc.) while still maintaining a same accuracy and ability to perform an operation on a target. The system 100 includes a table assembly 101, at least one rail assembly 120 coupled to the table assembly, and one or more manipulators 140 (also referred to as “repositionable structures”), coupled to each rail assembly 120 and controlled by one or more control boards 170. Each manipulator 140 can support one or more instruments 150, which can be removably or permanently mounted thereon. As shown in FIG. 1, the system 100 also can include the control system 1006, a user input and feedback system 1004, an output system 1005, and/or an auxiliary system 1008. In some embodiments, the system 100 is configured as a computer-assisted, teleoperable medical system, in which case table assembly 101 can be configured to support a patient (not shown) and the instruments 150 can be medical instruments. The system 100 in this configuration can be usable, for example, to perform any of a variety of medical procedures, such as surgical procedures, diagnostic procedures, imaging procedures, therapeutic procedures, etc. Moreover, the system 100, when configured as a teleoperable medical system, need not necessarily be used on a living human patient. For example, a non-human animal, a cadaver, tissue-like materials used for training purposes, and so on, can be supported on the table assembly 101 and worked on by system 100. In other embodiments, the system 100 is configured as a computer-assisted teleoperable system for use in non-medical contexts, in which case the table assembly 101 can be configured to support an inanimate workpiece (something being manufactured, repaired, tested, etc.) and the instruments 150 can be non-medical instruments, such as industrial instruments.

As shown in FIG. 1, the table assembly 101 includes a platform assembly 110 configured to support the patient, inanimate workpiece, or other such target; a support column 102 coupled to and supporting the platform assembly 110; and a base 105 coupled to the support column 102. The base can be configured to contact the ground or other surface upon which the table assembly 101 rests to provide stability for the table assembly 101. In some embodiments, the base 105 is omitted. In some embodiments, the base 105 includes mobility features, such as wheels, tracks, or other such features (not shown), to allow movement of the table assembly 101 along the ground or other surface. In FIG. 1, the support column 102 is illustrated as a single vertical columnar part to simplify the discussion, but the support column 102 could take any desired shape and could include any number of parts. For example, the support column 102 can include horizontal support structures (not illustrated) such as beams, rails, etc. to couple the platform assembly 110 to a vertical portion of the support column 102. Moreover, in various embodiments, the support column 102 can be telescoping and configured to extend and contract in height.

The platform assembly 110 includes one or more platform sections 103 to support the patient or workpiece. The platform sections 103 each have a support surface configured to contact and support the patient or workpiece. In some embodiments multiple platform sections 103 are used and the platform sections 103 are arranged in series to support different portions of the patient or workpiece. For example, in the embodiment illustrated in FIG. 1, the platform assembly 110 includes a first end section 103_1, one or more middle sections 103_2, and a second end section 103_3 (which may generally or collectively be referred to herein as “platform sections 103”), with the one or more middle sections 103_2 being arranged between the two end sections 103_1 and 103_3. In some embodiments, the first end section 103_1 can be configured to support a head of the patient, the second end section 103_3 can be configured to support the feet and/or legs of the patient, and the one or more middle sections 103_2 can be configured to support a torso and/or other portions of the patient. For convenience, the side of the platform assembly 110 that is near the first end section 103_1 (e.g., a left side in the orientation shown in FIG. 1) will be referred to herein as a “head” of the platform assembly 110 (or “head side” or “head end”) and the side of the platform assembly 110 that is near the second end section 103_3 (e.g., a right side in the orientation shown in FIG. 1) will be referred to herein as a “foot” of the platform assembly 110 (or “foot side” or “foot end”), but this is merely an arbitrary convention chosen herein for convenience of description and is not intended to limit the configuration or usage of the table assembly 101 (e.g., a head of a patient could be positioned at the “foot” side of the platform assembly 110 if desired, and vice versa). The relative positions of two components or of two portions of a single component can also be described using “head” and “foot” (e.g., a “head end” and a “foot end” of a rail 121) with “head” referring to the component or portion that is relatively closer to the head end of the table assembly 101 and “foot” referring to the component or portion that is relative closer to the foot end of the table assembly 101. In other embodiments, different numbers and arrangements of platform sections 103 are used, including one, two, four, or more platform sections 103. In some embodiments, one or more of the platform sections 103 can be movable relative to other platform sections 103 and/or relative to the support column 102. For example, in some embodiments, some or all of the platform sections 103 are coupled to adjacent platform sections 103 and/or to the support column 102 by rotatable joints such that at least some of the platform sections 103 can tilt relative to one another and/or relative to the support column 102. The platform assembly 110 can also be movable as a whole relative to the support column 102, as described in greater detail below.

The platform assembly 110 has a longitudinal dimension 198 (e.g., parallel to the x-axis in FIG. 1), a lateral dimension orthogonal to the longitudinal dimension (e.g., parallel to the y-axis in FIG. 1), and a thickness or height dimension orthogonal to both the longitudinal dimension 198 and lateral dimension (e.g., parallel to the z-axis in FIG. 1). As used herein, the longitudinal dimension 198 refers to a dimension of greatest extent of the platform assembly 110 when all of the platform sections 103 of the platform assembly are fully extended and all are oriented with their support surfaces roughly aligned in a same plane with one another (or when as close to this state as possible) so as to collectively form a combined support surface that is substantially planar with potentially small gaps between adjacent platform sections 103. In general, the longitudinal and lateral dimensions of the platform assembly 110 and the support surfaces of the platform sections 103 are oriented roughly parallel to the ground or other surface on which the table assembly 101 is supported when the platform assembly 110 is in a neutral configuration. However, one of ordinary skill in the art would understand that the platform assembly 110 as a whole and/or individual platform sections 103 thereof do not necessarily have to be parallel to the ground, and that one or both of the longitudinal and/or lateral dimensions can be tilted relative to the ground in various configurations through which the platform assembly 110 and/or platform section 103 can be movable, including in a neutral configuration in some cases. The platform assembly 110 and the various platform sections 103 thereof have various sides or faces that extend along the longitudinal dimension 198 or lateral dimension, and these can be referred to herein as longitudinally extending sides (or faces) and laterally extending sides (or faces), respectively. Specifically, a longitudinally extending side (or face) is a side (or face) of the platform assembly 110 or of a platform section 103 that extends along the longitudinal dimension 198 of the platform assembly 110 (i.e., along an x-direction in FIG. 1). For example, one longitudinally extending side 109b of the platform assembly 110 is indicated in FIG. 1. Similarly, a laterally extending side (or face) is a side (or face) of the platform assembly 110 or of a platform section 103 that extends along the lateral dimension of the platform assembly 110 (i.e., along a y-direction in FIG. 1). For example, two laterally extending sides 109a of the platform assembly 110 are indicated in FIG. 1. At least one of the platform sections 103 is directly coupled to and supported by the support column 102. The remaining platform sections 103 can be coupled directly to the support column 102 or they can be coupled indirectly to the support column 102 via a chain of one or more intervening platform sections 103. For example, in some embodiments a main platform section 103 (e.g., a middle section 103_2) is coupled to and directly supported by the support column 102 and the others of the platform sections 103 (e.g., end sections 103_1 and 103_3) are coupled to the main platform section 103 or to another platform section 103. As another example, in some embodiments, multiple platform sections 103 (all in some embodiments) are coupled directly to the support column 102 and not to another platform section 103.

In various embodiments, some or all of the above-described parts of the table assembly 101 can be movable relative to one another. For example, in some embodiments, the platform assembly 110 as a whole can be moved relative to the support column 102, such as by tilting around a horizontal axis, swiveling around a vertical axis, translating vertically along the support column 102, translating horizontally relative to the support column 102, and so on. In some embodiments, such movement of the platform assembly 110 as a whole can be provided by one or more joints that couple a main platform section 103 (e.g., a middle section 103_2) to the support column 102. Furthermore, as already noted above, individual platform sections 103 can be movable relative to one another and relative to the support column 102 as well, which can be facilitated by joints coupling the platform sections 103 to the support column 102 or to adjacent platform sections 103.

In some embodiments, the platform assembly 110 also includes one or more accessory rails 104. The accessory rails 104 can be configured to receive accessory devices removably mounted thereon, such as such as leg stirrups, liver retractors, arm boards, and bed extenders. In some embodiments, the accessory rails 104 adhere to industry standard specifications familiar to those of ordinary skill in the art to allow compatibility with accessory devices compliant with the standard. The accessory rails 104 can be attached to longitudinally extending side faces of one or more of the platform sections 103. One or more openings can be defined between an accessory rail 104 and the side face of the platform section 103 to which the accessory rail 104 is attached and portions of accessories mounted to the accessory rail 104 can be inserted through the openings.

As noted above, the manipulator system 100 also includes one or more manipulators 140, controlled by one or more control boards 170 (e.g., in conjunction with the control system 1006). While FIG. 1 illustrates two manipulators 140, any number of manipulators 140 can be included (such as, for example, one, two, three, or more manipulators mounted to each rail assembly 120, as described in further detail below). A manipulator 140 can include a kinematic structure of links coupled together by one or more joints. Specifically, the manipulators 140 each include a proximal link assembly including a proximal arm 141 movably coupled to the rail assembly 120 via one or more proximal arm joints 130, an intermediate link assembly including an intermediate arm 142 movably coupled to the proximal link assembly via one or more intermediate arm joints 145, and a distal link assembly including a distal arm 143 movably coupled to the intermediate link assembly by one or more distal arm joints 146. The distal link assembly can also include an instrument holding portion 169 coupled to the distal arm 143 and configured to carry the instrument 150. As sometimes used herein, portions of the instrument 150 may be referred to as an “instrument end effector” (or “instrument working portion”) and an “instrument shaft” to indicate a particular portion of the instrument 150.

The manipulators 140 are movable through various degrees of freedom of motion provided by various joints, including the proximal, intermediate, and distal arm joints 130, 145, and 146, thus allowing an instrument 150 mounted thereon to be moved relative to the worksite. Some of the joints can provide for rotation of links relative to one another, other joints can provide for translation of links relative to one another, and some can provide for both rotation and translation. In particular, in some embodiments, the proximal arm 141 is rotatably coupled to the rail 121 via a first proximal arm joint 130a, which provides for rotation of the proximal arm 141 relative to the rail 121 around a first axis 136 that is perpendicular to a longitudinal dimension 197 of the rail 121 (e.g., perpendicular to the x-direction in FIG. 1). In a neutral state of the proximal arm 141, the first axis 136 is also perpendicular to a lateral dimension of the rail 121 (e.g., perpendicular to the y-direction in FIG. 1), and thus in this state the first axis 136 is oriented vertically (i.e., perpendicular to the aforementioned horizontal plane, or in other words oriented in the z-direction in FIG. 1). In addition, in a neutral state of the table assembly 101, in which the platform assembly 110 is parallel to the ground and the rail 121 (i.e., an x-direction in the orientation of FIG. 1), the first axis 136 is also perpendicular to the longitudinal dimension 198 of the platform assembly 110, but this is not necessarily the case in other states (e.g., states in in which the platform assembly 110 is tilted relative to the rail 121, which can be possible in some embodiments).

In some embodiments, the proximal link assembly of certain manipulators 140 is configured to allow for rotation of the proximal arm 141 about a second axis 137, in addition to allowing for rotation about the first axis 136, with the second axis 137 being orthogonal to the first axis 136. In some embodiments, the rotation about the second axis 137 can be provided by a second proximal arm joint 130b included in the proximal link assembly. In particular, in some embodiments the proximal link assembly of certain of the manipulators 140 further includes a second proximal arm joint 130b, and the first and second proximal arm joints 130a and 130b together couple the proximal arm 141 to the rail 121, with the second proximal arm joint 130b providing for rotation of the proximal arm 141 relative to the rail 121 around a second axis 137 orthogonal to the first axis 136 and parallel to a longitudinal dimension 197 of the rail 121 (e.g., x-direction in FIG. 1). In some embodiments, the second proximal arm joint 130b is coupled between the rail 121 and the first proximal arm joint 130a, while in other embodiments the second proximal arm joint 130b is coupled between the first proximal arm joint 130a and the proximal arm 141 (not shown in FIG. 1). In still other embodiments, the rotation about the second axis 137 is provided by the first proximal arm joint 130a without the addition of a second proximal arm joint (e.g., the first proximal arm joint 130a is configured to provide rotation about multiple axes, such as a ball-and-socket joint). The longitudinal dimension 197 and, hence, the second axis 137 are parallel to the ground in some embodiments. In some embodiments, in the neutral state of the table assembly 101, the second axis 137 is also parallel to the longitudinal dimension 198 of the platform assembly 110. In some such embodiments, the second axis 137 is not parallel to the longitudinal dimension 198 of the platform assembly 110 in other states (e.g., states in in which the platform assembly 110 is tilted relative to the rail 121, which can be possible in some embodiments). Rotation of the proximal arm 141 around the second axis 137 (e.g., via the second proximal arm joint 130b) causes the proximal arm 141 to incline or decline relative to the horizontal plane, thus raising or lowering a distal end of the proximal arm 141 relative to the rail 121. In addition, as the proximal arm 141 inclines relative to the horizontal plane, movement of the proximal arm 141 can cause more distal portions of the manipulator 140 to correspondingly both raise and extend further across the table (as opposed to vertical movement alone).

In some embodiments, the rotation about the second axis 137 (e.g., via second proximal arm joint 130b) allows the proximal arm 141 to be moved between orientations ranging at least between a horizontal orientation and a vertical inclined orientation (e.g., at least 90 degrees of rotation). In some embodiments, rotation about the second axis 137 (e.g., via the second proximal arm joint 130b) can also allow for rotation of the proximal arm 141 to orientations that are declined relative to a horizonal orientation. In some embodiments, certain manipulators 140 are provided with the ability to rotate about the second axis 137 (e.g., via the second proximal arm joint 130b) while others are not. For example, in some embodiments a first manipulator 140 whose proximal arm 141 is positionable under a second manipulator 140 in a nested configuration (e.g., as described in more depth below) can be provided with the second proximal arm joint 130b (e.g., because the lower positioning of the proximal arm 141 makes room for the proximal arm joint 130b), while a second proximal arm joint 130b can be omitted in the second manipulator 140 (e.g., because the higher positioning of the proximal arm 141 of the second manipulator 140 does not leave sufficient room for the second joint). In other embodiments (not illustrated), coupled to a same rail 121 all of the manipulators 140 (or all manipulators 140 in the system 100, in some embodiments) are provided with the ability to rotate about the second axis 137 (e.g., via second proximal arm joints 130b). In still other embodiments (not illustrated), none of the manipulators 140 coupled to a given rail 121 (or none of the manipulators 140 in the entire system 100, in some embodiments) are provided with the ability to rotate about the second axis 137.

In addition, in some embodiments, the proximal arm 141 is extendable and retractable. For example, the proximal arm 141 can include two or more links that are translatable relative to one another in a telescoping fashion to extend or retract the proximal arm 141. In other words, these two or more links are coupled together by, or they themselves form, a prismatic joint. For example, in some embodiments the proximal arm 141 includes an outer link that has a bore (for example an axial bore extending along a longitudinal axis of the proximal arm 141) and an inner link that is nested within the outer link in the bore thereof.

In addition, in some embodiments, the proximal arm 141 has an asymmetrical shape, meaning that, in extending from a proximal end portion of the proximal arm 141 to a distal end portion of the proximal arm 141, the proximal arm 141 follows a non-straight path (i.e., a path that deviates from a hypothetical straight line extending between (e.g., connecting) the two end portions). More specifically, the proximal arm 141 can extend between the proximal arm joint 130 coupled to the proximal end portion of the proximal arm 141 and an intermediate arm joint 145 (e.g., described in more depth below) coupling the distal end portion of the proximal arm 141 to intermediate arm 142, with a centerline of the proximal arm 141 extending between these joints 130 and 145 deviating from a straight line between respective axes of the joints 130 and 145. For example, in some embodiments the proximal arm 141 has a smoothly curved shape (e.g., a centerline of the proximal arm follows a smoothly curved path), while in other embodiments the proximal arm 141 has a segmented shape including multiple straight and/or curved segments joined together at angles (e.g., an L-shape).

In some embodiments, the intermediate arm 142 can be rotatably coupled to the distal end portion of the proximal arm 141 via one or more intermediate rotary joints 145. For example, the intermediate arm joints 145 can provide for rotation of the intermediate arm 142 relative to the proximal arm 141 about a third axis (not illustrated) perpendicular to the intermediate arm 142 and the proximal arm 141. In addition, in some embodiments, the intermediate arm joints 145 can provide for rotation of a distal end of the intermediate arm 142 relative to the proximal arm 141 about an axis that is parallel to a longitudinal dimension of the intermediate arm 142. In some embodiments, the intermediate arm 142 is also extendable and retractable. For example, the intermediate arm 142 can include two or more links that are translatable relative to one another in a telescoping fashion to extend or retract the intermediate arm 142, in a manner similar to that described above in relation to proximal arm 141. In some embodiments, the links of the intermediate arm 142 are both translatable relative to one another along a longitudinal dimension of the intermediate arm 142 and also rotatable relative to one another about an axis parallel to the longitudinal dimension of the intermediate arm 142, thus providing for the above-described rotation of the distal end of the intermediate arm 142 relative to the proximal arm 141 about the axis that is parallel to a longitudinal dimension of the intermediate arm 142.

Moreover, in some embodiments, the distal arm 143 is movably coupled to the instrument holding portion 169 via a wrist 147, which includes joints for moving the instrument holding portion 169 relative to the distal arm 143. The joints of the wrist 147 can be referred to herein as wrist joints. In some embodiments, the wrist 147 provides multiple rotational degrees of freedom motion. For example, in some embodiments the wrist 147 has three rotational degrees of freedom of motion for the instrument holding portion 169 relative to the distal arm 143. For example, the wrist 147 can be rotatably coupled to the distal arm 143 to provide a roll degree of freedom of motion including rotation of the wrist 147 as a whole about an axis parallel to the distal arm 143, and the wrist 147 can further include two joints for providing yaw and pitch degrees of freedom of motion including rotation around pitch and yaw axes which are perpendicular to one another. One of the pitch and yaw axes is also perpendicular to the roll axis (the other of the pitch and yaw axes can also be perpendicular to the roll axis in a neutral state of the wrist 147, but not necessarily in other states). In some embodiments, the joints providing some of the degrees of freedom of motion of the wrist 147 (e.g., yaw and pitch, in some embodiments) are driven by actuators disposed remotely from the wrist 147, such as in a more proximal portion of the manipulator 140 with actuation elements (such as cables, filaments, belts, bands, linkages, etc.) extending from the actuators to the wrist 147 to drive the motion of the wrist. For example, in some embodiments, the wrist includes two wrist joints disposed in the wrist that provide rotation about the yaw and pitch axes, and these two wrist joints can be coupled to actuation elements (e.g., cables) that drive the rotation. In some embodiments, the actuators that drive the wrist 147 are positioned in the distal arm 143. Disposing the actuators remotely from the wrist 147 allows the wrist 147 to be more compact. Wrists that are compact, such as the wrists 147, can be positioned more closely to portions of other manipulators 140, in some circumstances, which can allow for greater flexibility in the positioning and posing of the manipulators 140. Moreover, placing the actuators in a more proximal portion of the manipulators 140, such as in the distal arm 143, moves the weight of the actuators closer to a proximal end of the kinematic chain that makes up the manipulator 140, thus reducing the moment arm (leverage) created by the weight of the actuators.

Some or all of the joints of the system 100 described above (as well as other joints that might be present in the system) can be powered joints, meaning a powered drive element (also referred to as a “driver” herein) can control movement of the joint through the supply of motive power. Such powered drive elements can include, for example, electric motors, pneumatic or hydraulic actuators, and other types of powered drive elements those having ordinary skill in the art would be familiar with. In some embodiments, the joints of the wrist 147 are powered joints. Additionally, in some embodiments some of the joints of the system 100 can be manually articulable (e.g., unpowered) joints, which can be articulated manually for example by manually moving the links coupled thereto. Joints referred to herein as unpowered can lack powered drive elements to drive articulation of the joint but still can include other powered aspects or devices, such as electronically (or hydraulically/pneumatically, etc.) controlled brakes, sensors (e.g., position, velocity, force, torque sensors), or other powered devices. Additionally, in some embodiments some of the joints of the system 100 can be partially powered and partially manually articulable—for example powered elements such as motors can assist manipulation, such as by compensating for gravity loads, friction, etc., but some manual force input can also be used to cause the articulation. Additionally, some joints (whether powered or not) can also be passively counterbalanced (e.g., via masses or springs). Certain joints can be actively controllable during performance of a procedure, for example, under the control of one or more control boards 170 communicatively coupled to a control system 1006 and in response to inputs recited at a user input and feedback system 1004. Other joints, sometimes referred to as setup joints, can be articulated during a setup phase in preparation for performance of the procedure but can generally remain more-or-less stationary during performance of the procedure. Setup joints can be powered, manually articulable, or partially powered. For example, in some embodiments, the proximal arm joints 130 and the prismatic joint that provides extension of the proximal arm 141 are setup joints.

As noted above, the instrument holding portion 169 is configured to support an instrument 150, and in some embodiments the instrument holding portion 169 includes a drive interface to removably couple the instrument 150 and to provide driving inputs (e.g., mechanical forces, electrical inputs, etc.) to drive the instrument 150. For example, the drive interface can include output couplers (not illustrated) to engage (directly or indirectly via an intermediary) with input couplers (not illustrated) of the instrument 150 to provide driving forces or other inputs to the mounted instrument 150 to control various degree of freedom movement and/or other functionality of the instrument 150, such as moving an end-effector of the instrument, opening/closing jaws, driving translation and/or rotation of a variety of components of the instrument, delivery of substances and/or energy from the instrument, and various other functions those of ordinary skill in the art are familiar with. The output couplers can be driven by actuators (e.g., electrical servo-motors, hydraulic actuators, pneumatic actuators) with which those of ordinary skill in the art have familiarity. An instrument sterile adaptor (ISA) can be disposed between the instrument 150 and the instrument manipulator mount interface to maintain sterile separation between the instrument 150 and the manipulator 140. The instrument manipulator mount can also include other interfaces (not illustrated), such as electrical interfaces to provide and/or receive electrical signals to/from the instrument 150. The instruments 150 can include any tool or instrument, including, for example, industrial instruments and medical instruments (e.g., surgical instruments, imaging instruments, diagnostic instruments, therapeutic instruments, etc.). In some embodiments, the system 100 can include flux delivery transmission capability as well, such as, for example, to supply electricity, fluid, vacuum pressure, light, electromagnetic radiation, etc. to the end effector. In other embodiments, such flux delivery transmission can be provided to an instrument through another auxiliary system 1008, described further below and as those of ordinary skill in the art would be familiar with in the context of computer-assisted, teleoperated medical systems.

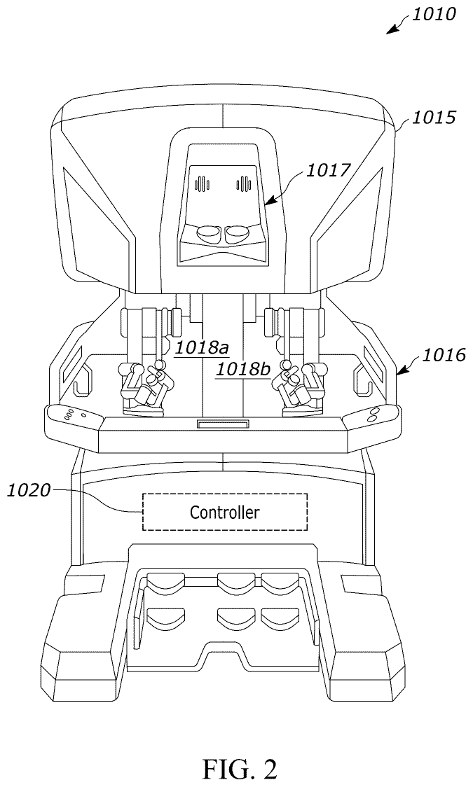

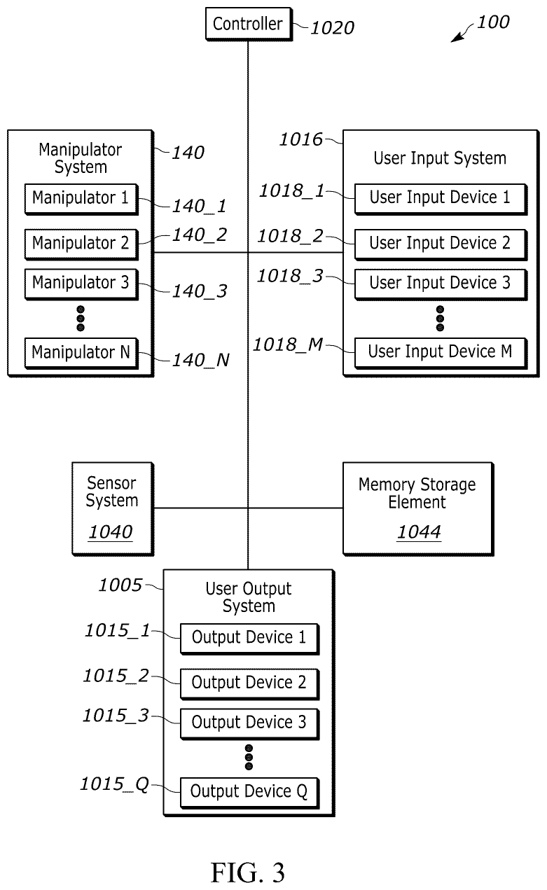

Additional details relating to the manipulators are described below with reference to FIGS. 2 and 3, which illustrate various embodiments of the manipulators 140. Moreover, in some embodiments, aspects of the manipulators 140 can be similar to the manipulators described in U.S. Provisional Patent Application No. 63/336,773, entitled “RAIL ASSEMBLY FOR TABLE MOUNTED MANIPULATOR SYSTEM, AND RELATED DEVICES, SYSTEMS AND METHODS,” inventor Ryan Abbott, filed Apr. 29, 2022; in U.S. Provisional Patent Application No. 63/336,778, entitled “NESTING PROXIMAL LINKS FOR TABLE MOUNTED MANIPULATOR SYSTEM, AND RELATED DEVICES, SYSTEMS AND METHODS,” first named inventor Bram Lambrecht, filed Apr. 29, 2022; or those described in, for example, U.S. Pat. No. 9,358,074 (filed May 31, 2013) to Schena et al., entitled “Multi-Port Surgical Robotic System Architecture,” U.S. Pat. No. 9,295,524 (filed May 31, 2013) to Schena et al., entitled “Redundant Axis and Degree of Freedom for Hardware-Constrained Remote Center Robotic Manipulator,” and U.S. Pat. No. 8,852,208 (filed Aug. 12, 2010) to Gomez et al., entitled “Surgical System Instrument Mounting,” WO International Publication Number 2023/212344 A1 (filed Apr. 28, 2023) entitled “TABLE-MOUNTED MANIPULATOR SYSTEM, AND RELATED DEVICES, SYSTEMS AND METHODS,” first named inventor Steven Manuel, the contents of each of which are incorporated herein by reference in their entirety. Various other embodiments of manipulators can include those as configured as part of the medical systems that are part of various da Vinci® Surgical Systems, such as the da Vinci X®, da Vinci Xi®, and da Vinci SP systems, commercialized by Intuitive Surgical, Inc., of Sunnyvale, California.

The number, locations, and types of links, joints, and control boards of the manipulators, as well as the various degrees of freedom of motion thereof, are not limited to those described above. In some embodiments, manipulators include additional links, joints, control boards, and/or degrees of freedom beyond those described above. In other embodiments, manipulators can omit certain of the links, joints, control boards, and/or degrees of freedom described above. Embodiments contemplated herein include embodiments including various combinations of one or more of the links, joints, and degrees of freedom of motion described above.

In some embodiments, the rail assembly 120 can be similar to the rail assemblies described in U.S. Provisional Patent Application No. 63/336,773, entitled “RAIL ASSEMBLY FOR TABLE MOUNTED MANIPULATOR SYSTEM, AND RELATED DEVICES, SYSTEMS AND METHODS,” in U.S. Provisional Patent Application No. 63/336,778, entitled “NESTING PROXIMAL LINKS FOR TABLE MOUNTED MANIPULATOR SYSTEM, AND RELATED DEVICES, SYSTEMS AND METHODS,” and in WO International Publication Number 2023/212344 A1 (filed Apr. 28, 2023) entitled “TABLE-MOUNTED MANIPULATOR SYSTEM, AND RELATED DEVICES, SYSTEMS AND METHODS,” first named inventor Steven Manuel, each incorporated by reference above.

The user input and feedback system 1004, output system 1005, control system 1006, and auxiliary system 1008 will be further described. Some or all of these components can be provided at a location remote from the table assembly 101. The user input and feedback system 1004 is operably coupled to the control system 1006 and includes one or more input devices to receive input control commands to control operations of the manipulators 140, instruments 150, rails assembly 120, and/or table assembly 101. Such input devices can include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device can be provided with the same degrees of freedom as the associated instrument that they control, and as the input device is actuated, the instrument, through drive inputs from the manipulator assembly, is controlled to follow or mimic the movement of the input device, which can provide the user a sense of directly controlling the instrument. Telepresence input devices can provide the operator with telepresence, meaning the perception that the input devices are integral with the instrument. The user input and feedback system 1004 can also include feedback devices, such as a display device (not shown) to display images (e.g., images of the workspace as captured by one of the instruments 150), haptic feedback devices, audio feedback devices, other graphical user interface forms of feedback, etc.

The user output system 1005 may include one or more monitors, screens, headsets, mobile devices, etc. used to display information to a user. For example, the user output system 1005 may display an actual or virtual representation of an instrument 150 as described in more detail further herein.

The control system 1006 can control operations of the system 100. In particular, the control system 1006 can send control signals (e.g., electrical signals) to the table assembly 101, rail assembly 120, manipulators 140, control boards 170, and/or instruments 150 to control movements, provide status indications, and/or perform other operations of the various parts. In some embodiments, the control system 1006 can also control some or all operations of the user input and feedback system 1004, the output system 1005, the auxiliary system 1008, or other parts of the system 100. The control system 1006 can include an electronic controller to control and/or assist a user in controlling operations of the manipulators 140, and other components of the system 100. The electronic controller includes processing circuitry configured with logic for performing the various operations. The logic of the processing circuitry can include dedicated hardware to perform various operations, software (machine readable and/or processor executable instructions) to perform various operations, or any combination thereof. In examples in which the logic includes software, the processing circuitry can include a processor to execute the software instructions and a memory device that stores the software. The processor can include one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In cases in which the processing circuitry includes dedicated hardware, in addition to or in lieu of the processor, the dedicated hardware can include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry can also include any combination of dedicated hardware and processor plus software.

Differing degrees of user control versus autonomous control can be utilized in the system 100, and embodiments disclosed herein can encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 can execute pre-programmed logic (e.g., a software program) and can determine and send control commands based on the programming (e.g., in response to a detected state or stimulus specified in the programming). In some systems, some operations can be user controlled and others autonomously controlled. Moreover, some operations can be partially user controlled and partially autonomously controlled—for example, a user input command can initiate performance of a sequence of events, and then the control system 1006 can perform various operations associated with that sequence without needing further user input.

Additionally, the control system 1006 may analyze kinematic data associated with the manipulator system 100 and/or the components thereof (e.g., gathered from one or more kinematic data sensors (not shown)) to determine a pose of the various components with respect to forces such as gravity, friction, etc. For example, in some embodiments, the control system 1006 may register the manipulators 140 and/or the portions thereof with respect to a coordinate system (such as those described elsewhere herein). The control system 1006 may determine a metric based on the kinematic data (e.g., friction, gravitational force, torque, etc.) for performing various operations as described herein.

As illustrated in FIG. 1, some or all of the manipulators 140 may be in various deployed states. The deployed states include states in which one or more of the manipulators 140 are not stowed, which in some embodiments means at least that the one or more manipulators 140 are at least partially unfolded/uncompacted while remaining coupled to the rail 121 and removed from a stowed location (e.g., removed out from under the platform assembly 110). The manipulators 140 can be positioned in a variety of deployed states.

During operation, the control system 1006 monitors kinematic data and positioning data associated with manipulators 140, for example, to detect the location of a manipulator and/or determine an alternate approach as described herein. Depending on the embodiment, the control system 1006 may obtain the kinematic data from one or more kinematic sensors (e.g., motion sensors, acceleration sensors, force sensors, etc.) associated with one or more components of the manipulators 140 (e.g., links, joints, etc.).

FIG. 2 illustrates an embodiment of an operator console 1010 that may be implemented in the system 100 of FIG. 1. In some embodiments, the manipulators 140 are teleoperated by an operator through a user input system 1016 physically separate from the manipulators 140. In some implementations, the manipulators 140 can also be controlled directly through manual interaction with the manipulators 140 themselves (e.g., in a clutch operation mode as described herein). In some embodiments, the operator teleoperates the manipulators 140 and monitors instruments supported by the manipulators 140 using a user input system (e.g., user input and feedback system 1004) and a user output system (e.g., user output system 1005). In some examples, the console 1010 is a standalone console that includes the user input system 1016 (including or part of, in various embodiments, the user input and feedback system 1004) and/or the user output system 1005 (including or part of, in various embodiments, the user output system 1005 of FIG. 1). In the example shown, the console 1010 includes user input system portions such as input devices 1018a, 1018b, and a user output system comprising a stereoscopic display device 1017. In further embodiments, the display device 1017 is a monitor-type display device 1017 that provides monoscopic or 3D images in various implementations.

In some examples, a non-console based user input and output system (not shown) may be used in addition to and/or alternatively to the console 1010. In some such embodiments, the system includes a user input system (e.g., user input system 1004) comprising handheld user input devices (not shown). The handheld user input device not physically constrained by links and joints to a base or console such as console 1010. In some such embodiments, the user input system 1016 further includes a sensor system (not shown) that communicates with one or more input devices 1018 for detecting user input.

When the system 100 is operated in a following mode, the operator can operate the user input system 1016 to generate a set of user input signals to control motion of the manipulators 140. In some embodiments, controller 1020 is physically located with the console 1010. However, controller 1020 may be physically separate from the console 1010 and communicate with components of the console 1010 via electronic signals transmitted via wired or wireless technology.

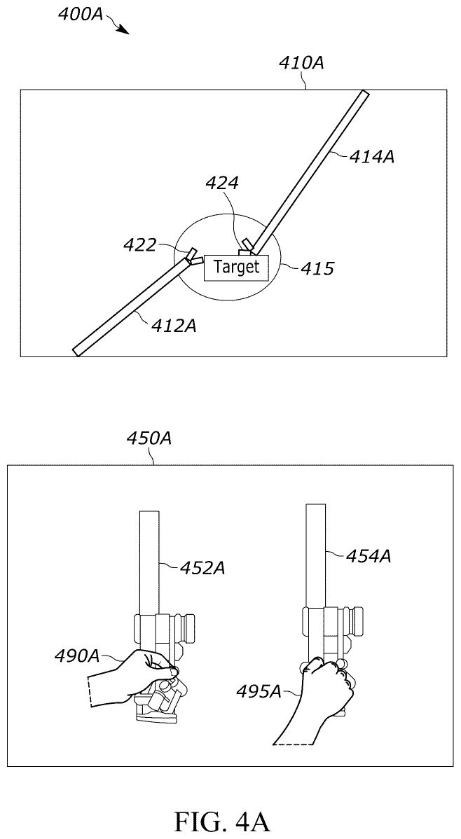

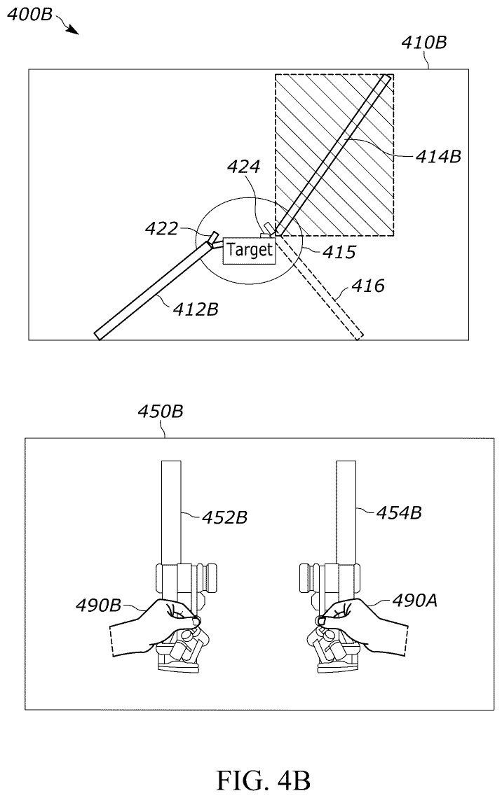

During operation of the system 100, the operator can view the display device 1017 to view imagery (e.g., two-dimensional imagery or three-dimensional imagery), representing the instruments mounted on the manipulators 140 while the manipulators 140 are being controlled by the operator. For example, an instrument 150 that is coupled to an image capture device, such as a camera, mounted to one of the manipulators 140. The image capture device generates imagery of distal end portions of other instruments 150 mounted to the other manipulators 140. During operation of the system 100, the operator can monitor poses of distal end portions of the instruments 150 using the imagery presented on the display device 1017. In some embodiments, as described with more detail below with regard to FIGS. 4A and 4B, the display device 1017 may display image data that was generated in addition to and/or in place of the actual image data generated by the image capture device(s). For example, the display device 1017 may obfuscate the image data depicting the manipulators 140 and/or instrument associated with the manipulators 140 and generate a virtual representation of the manipulators 140 and/or instrument(s) 150 for display to the operator. In some such embodiments, the display device 1017 generates the virtual representation such that at least part of the manipulator 140 and/or associated instrument 150 is depicted in a different location and/or pose than the actual manipulator 140 and/or associated instrument 150 as represented in the image data generated by the image capture device(s).

The user input system 1016 is operably connected to the manipulators 140 (e.g., wirelessly or using a wired connected). As depicted, the user input system 1016 includes multiple distinct portions operable by the operator to control operations of the manipulators 140. The user-operable portions, in some cases, correspond to distinct user input devices 1018 that generate distinct control signals based upon their respective motion. In the illustrated embodiment, the user input system 1016 includes manually operable user input devices (e.g., input devices 1018a, 1018b) (collectively referred to as user input devices 1018) corresponding to the user-operable portions of the user input system 1016 and movable relative to the manipulators 140 to control movement of the manipulators 140. It should be appreciated that this relative motion to control movement of the manipulators 140 may cause the motion required to perform certain actions to strain the operator and/or otherwise approach or exceed the operator's physical range of motion for manipulating the user input devices 1018. Additionally, the user input system 1016 can include other user input devices (e.g., keyboards, touchscreens, buttons, foot pedals, etc.) in addition to the user input devices 1018. The other user-operable portions can be used to interact with a user interface presented on the display device 1017 and/or otherwise allow user control of other operations of the system 100.

Referring to FIG. 3 and as described herein, an example system diagram representative of the system 100 for generating and displaying a virtual representation of one or more manipulator(s) and/or instrument(s) includes the manipulator 140, the controller 1020, a user output system 1005, and the user input system 1016. It will be understood that, in some embodiments, as shown in FIG. 3, the manipulator 140 can include any number of manipulators. For example, the manipulator 140 includes N manipulators (e.g., Manipulator 1 through Manipulator N, collectively referred to as manipulators 140). Similarly, depending on the embodiment, as shown in FIG. 3, the user input system 1016 includes any number of user input devices 1018 or user-operable portions of the user input system 1016. For example, the user input system 1016 includes M user input devices (e.g., User Input Device 1 through User Input Device M, collectively referred to as user input devices 1018). The user input system 1016 may include the user input devices 1018 as described with respect FIG. 2, and may additionally include: joysticks, touchscreens, gloves, foot pedals, touchscreens, or handheld remotes. The controller 1020 may operate in a pairing mode to pair a particular user input device 1018 with a particular manipulator 140 such that the particular manipulator 140 is controlled based upon user interaction with the particular user input device 1018.

In some implementations, the system 100 includes a sensor system 1040. The sensor system 1040 includes sensors operable to detect movement of the user input devices 1018. The sensor system 1040 can detect poses (e.g., positions, orientations, or both positions and orientations) of the user input devices 1018 and the manipulators 140 within a field of view (FOV). Sensors of the sensor system 1040 include, for example, stereoscopic image sensors, infrared sensors, ultrasonic sensors, image capture devices, accelerometers, position encoders, optical sensors, or other appropriate sensors for detecting motion and poses of the manipulators 140 and the user input devices 1018.

The user output system 1005 provides human-perceptible feedback to the operator and includes Q user output devices (e.g., User Output Device 1 through User Output Device Q). Depending on the embodiment, the user output devices 1015 can include various monitors, screens, mobile devices communicatively coupled to the controller 1020, etc. (e.g., the display device 1017, a mobile phone communicatively coupled to the controller 1020, an extended reality headset, etc.). The feedback provided by the user output system 1005 can include feedback provided during the following mode, during a calibration process, during a pairing process, etc. to provide guidance to the operator for controlling the manipulators 140. Furthermore, the user output system 1005 is operable to present an actual or virtual representation of the manipulators 140 and/or instrument 150 coupled therewith (e.g., on the display device 1017). In some implementations, the user output system 1005 and the user input system 1016 are implemented at the console 1010.

The system 100 can further include a memory storage element 1044. The memory storage element 1044 can store data indicative of pairings between the manipulators 140 and the user input devices 1018, image data gathered from the sensor system 1040, one or more algorithms and/or models for generating the virtual representation of the manipulators 140, one or more user preferences regarding poses and/or pose history, etc.

FIG. 4A depicts an example instrument positioning 400A including an endoscopic view 410A and a controller positioning view 450A corresponding to the endoscopic view 410A. In the exemplary embodiment of FIG. 4A, the endoscopic view 410A may represent image data generated by an endoscope instrument 150, whereas the controller positioning view 450A is merely illustrative of controller positioning at the time the image data included in the endoscopic view 410A was generated. Accordingly, it should be appreciated that the controller positioning view 450A may not represent a set of image data and may not be displayed on a display device 1005.

As illustrated, the endoscopic view 410A depicts a target 415 and two instrument end effector representations 422 and 424 coupled to two instrument shaft representations 412A and 414A. Depending on the embodiment, the endoscopic view 410A may be a stream of image data (e.g., from an imaging device such as a stereoscopic camera), and the instrument end effector representations 422 and 424 may be views of instruments 150 present in the stream of image data. In further embodiments, the endoscopic view 410A may be a data stream form of image data received from an endoscopic instrument, and the instrument shaft representations 412A and 414A may be generated representations that are generated and/or modified additional image data that forms the data stream being received. In still further embodiments, the endoscopic view 410A may be a generated view using delayed and/or past image data, and the instrument shaft representations 412A and 414A may similarly be generated based on such. It will be understood that, although the instrument positioning references an “endoscopic view” 410A, the view 410A may include or be additional or alternate views (e.g., a target view, an outside view, etc.) and may be generated by other types of image sensors. Therefore, the term “endoscopic view” should not be construed to be limited to image data generated by an endoscope unless explicitly indicated otherwise. Similarly, although the exemplary embodiment of FIG. 4A includes two instrument shaft representations 412A and 414A, it will be understood that more or fewer instrument shaft representations may be included.

The controller positioning view 450A depicts the positioning of the controllers 452A, 454A (e.g., the user input devices 1018 of FIGS. 1-3) to cause the instruments 150 to be depicted in the manner represented by the instrument end effector representations 422 and 424 and the instrument shaft representations 412A and 414A in the endoscopic view 410A. In particular, the positioning of controller 452A corresponds to the instrument end effector representation 422 and the instrument shaft representation 412A, and the positioning of controller 454A corresponds to the instrument end effector representation 424 and the instrument shaft representation 414A.

In the exemplary embodiment of FIG. 4A, conventional approaches for interpreting motion of the controllers 452A, 454A based on the instrument shaft representations 412A and 414A, respectively, are implemented. That is, the control system 1006 (e.g., via the controller 1020) may interpret motion of the controllers 452A and 454A based on the pose of the instrument shaft representations 412A and 414A (and, correspondingly, the instrument end effector representations 422 and 424), respectively. While the conventional approach is suitable for many scenarios, in the illustrated scenario, an operator must contort the controller 454A at sharp angles and/or directions that approach, or even exceed, the operator's physical range of motion to manipulate the instrument represented by the instrument shaft representation 414A in a manner needed to perform a procedure. For example, operator limbs 490A and 495A illustrate an example posture the operator must take to reach the appropriate positioning of the instrument shaft representations 412A and 414A in the endoscopic view 410A. The operator must then maintain the positioning of the controller 454A while performing the procedure, which may be long, strenuous, and/or otherwise physically demanding on the operator, and lead to imprecise operation of the instruments 150.

FIG. 4B illustrates an alternative example instrument positioning 400B to example instrument positioning 400A. Similar to the endoscopic view 410A, the endoscopic view 410B includes a target and two instrument shaft representations 412B and 414B. The endoscopic view 410B also includes a virtual instrument shaft representation 416. The endoscopic view 410B additionally includes the instrument end effector representations 422 and 424 in the same positioning as in FIG. 4A.

In response to detecting a trigger event, the control system 1006 may operate in a different mode of operation, in which the control system 1006 causes the endoscopic view 410B to display the virtual instrument shaft representation 416 and instrument end effector representation 424 of a particular instrument 150 in place of the instrument shaft representation 414B representative of the actual physical location of the particular instrument 150. In particular, the system 100 may use image data (e.g., from a stereoscopic imaging device) to generate image data representative of the area obscured by the instrument shaft representation 414B and overlay the generated image data onto the region of the endoscopic view 410B associated with instrument shaft representation 414B such that the instrument shaft representation 414B is no longer visible. In further embodiments, the system 100 may use an in-painting model to generate the image data used to obfuscate the instrument shaft representation 414B.

The controller 1020 may determine the pose of the virtual instrument shaft representation 416 using a predicted position, a preferred pose (e.g., based on an ergonomic and/or otherwise comfortable position for a user), a user indication, a calibration process, etc. In some such embodiments, the control system 1006 converts signals from a user input (e.g., as described above with regard to FIGS. 2 and 3) to determine a desired movement and/or positioning to mirror appropriate movements of the actual instrument shaft based on what the movements the user indicates for the virtual instrument shaft representation 416. In further embodiments, the control system 1006 generates the virtual instrument shaft representation 416 in addition to the instrument shaft representation 414B as a guide for a more comfortable positioning for the user.

Unlike the conventional mode of operation associated with FIG. 4A, the controller 1020 now interprets motion of the controller 454B as if the instrument shaft to which the controller 454B is paired is posed in accordance with the virtual instrument shaft representation 416 as opposed to the instrument shaft representation 414B. Accordingly, the controller 1020 may be configured to convert control signals of the controller 454B that would normally be referenced with regard to the instrument shaft representation 414B to instead be referenced with respect to the virtual instrument shaft representation 416 when generating the actual control commands that control the pose of the instrument 150 via the instrument shaft. As a result, as shown in the controller positioning 450B, the positioning for controller 454B is more ergonomic for the operator, resulting in casier control of the instrument and instrument shaft to perform the procedure. This allows for a greater mobility and range of action without compromising the ability of the user to safely perform the procedure. In some embodiments, the controller 1020 generates, displays, and/or obscures the instrument shaft representations 412B, 414B, and/or 416 while maintaining the instrument end effector representations 422 and 424. As such, the actual instrument display(s) remain unaltered while the shaft representation(s) are moved at the wrist to depict a more ergonomic view. Furthermore, a user is able to better position the controllers 452B and/or 454B (e.g., more ergonomically, for a better view, more efficiently, etc.) without impeding any view of the instrument end effector, mitigating and/or preventing risk of accidental damage to the target 415. For example, in FIG. 4B, operator limbs 490B and 495B are positioned more ergonomically compared to operator limbs 490A and 495A while maintaining the positioning of instrument end effector representations 422 and 424.

As described above, the control system 1006 may switch from the first mode of operation depicted in FIG. 4A to the second mode of operation depicted in FIG. 4B in response to detecting a trigger event. In some such embodiments, the trigger event is an output of one or more safety checks. For example, the control system 1006 may generate the virtual instrument shaft representation 416 responsive to: (i) determining that a portion of the instrument 150 and/or manipulator 140 corresponding to the instrument shaft representation 414B has not collided and/or will not collide (e.g., risk meeting a predetermined threshold value) with anything in the operation area, (ii) determining that a portion of the instrument 150 and/or controller 454A corresponding to the instrument shaft representation 414A is not within a predetermined range of a motion limit associated with the instrument and/or operator, (iii) determining that the operator is in control of the manipulator(s) 140 (e.g., responsive to a manual indication to switch modes, monitoring of the instrument and/or instrument motion, etc.), and/or any other such safety-related indication.

In further embodiments, the control system 1006 generates the pose (e.g., position, location, angle, etc.) based on one or more factors. For example, in various embodiments, the control system 1006 generates the pose based on at least one of a position at which continued operation of the controller 454B is ergonomically friendly, a specific position as indicated by an operator, a central position (e.g., a neutral position) relative to the workspace in which the user works, etc.

While the foregoing relates to case of operation and/or safety trigger events, in further embodiments, the control system 1006 utilizes trigger events based on visibility. For example, the control system 1006 may determine that the instrument shaft representation 414B is obscuring the target 415. In response, the control system 1006 may generate the virtual instrument shaft representation 416 such that the user is provided with a view of the target 415. It should be appreciated that visibility of the target 415 may also be considered as a factor when determining the pose of the virtual instrument shaft representation 416 when detecting an ergonomics-based trigger event. In still other embodiments, the user may provide and/or calibrate one or more rules for generating the virtual instrument shaft representation 416 and/or for priority in generating the virtual instrument shaft representation 416.

In further embodiments, the control system 1006 utilizes trigger events associated with a user indication. For example, the control system 1006 may receive an indication from the user (e.g., from an input device 1018) to enable the control system 1006 to utilize the virtual instrument shaft representation 416. In still further embodiments, the control system 1006 may utilize one or more pre-programmed and/or customized triggers by a user. For example, a user may customize the control system 1006 to generate and display the virtual instrument shaft representation 416 responsive to detecting that the controller 454B is held at a predetermined angle for a predetermined period of time, determining a likelihood that the controller 454B will be held at a predetermined angle for a predetermined period of time, detecting or receiving an indication of a predetermined type of procedure, etc.

In still further embodiments, the control system 1006 generates warning messages responsive to one or more indications associated with the virtual instrument shaft representation 416. For example, the control system 1006 may generate a notice to a user that the virtual instrument shaft representation 416 will be generated upon enabling or triggering the virtual representation. In further embodiments, the control system 1006 generates a notice to a user that one or more movement options are disabled (e.g., moving elements of the manipulator 140 associated with the instrument), such as in response to detecting an actual position of the instrument 150 and/or manipulator 140 near an object. As another example, the control system 1006 may generate a notification a user is approaching a range of motion limit associated with a shaft and/or wrist of an instrument 150 and/or the controller 454B. In still further embodiments, the system 100 determines that a range of motion limit is being approached based on a calibration process, using one or more feedback sensors associated with the manipulator(s) 140 and/or instrument, based on user preferences, etc. For example, the control system 1006 may require an initial calibration process associated with a workspace, user, target, etc. to determine one or more bounds or limitations (e.g., a range of motion limit for the user, an area in which to prevent movement within (e.g., close to other manipulators or instruments, close to personnel, etc.), an area in which to provide notifications, etc.).

In some embodiments, the control system 1006 generates different versions of the endoscopic views (e.g., endoscopic views 410A and 410B) for different display devices. For example, the control system 1006 may display the endoscopic view 410A depicting the actual representations of the instrument shafts (e.g., instrument shaft representations 412A and 414A) on some display devices while depicting the endoscopic view 410B that includes the virtual instrument shaft representation 416 on other display devices. As such, different personnel may see the appropriate endoscopic view 410 needed to perform their role in the procedure (e.g., a surgeon display device presents the endoscopic view 410B to support ergonomic control and a supervising attendee display device presents the view 410A to observe the actual performance of the procedure).

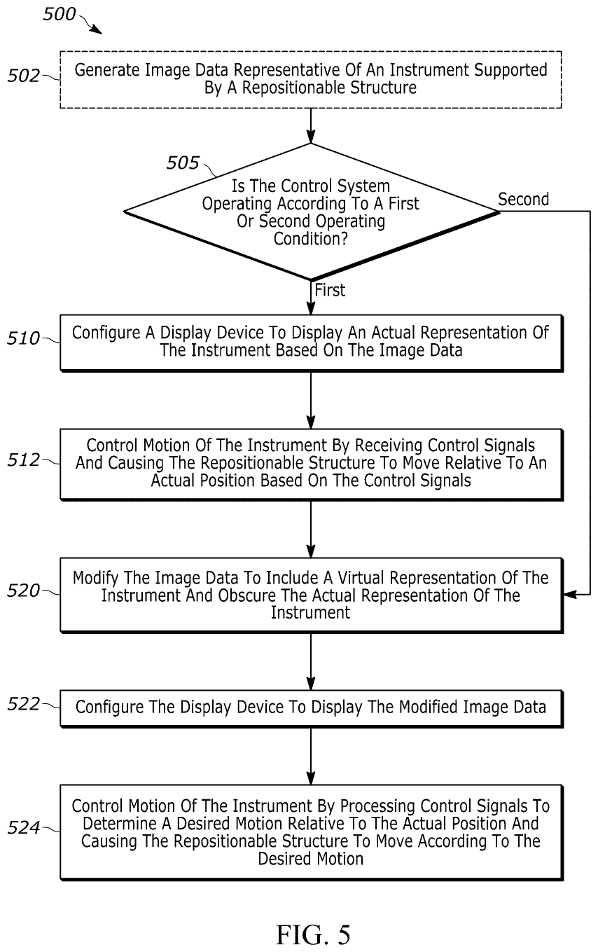

FIG. 5 is an example flow diagram of an example method 500 for operating a repositionable structure associated with one or more instruments and display a view of the instrument shafts and/or end effectors to an operator, performed by, for example, a system with manipulators according to any of FIGS. 1-4B. An example system for performing the method 500 may include a repositionable structure such as the manipulators 140, an instrument such as the instrument 150, and an imaging device (e.g., an endoscopic camera) as described herein. The system further includes a controller, such as the control system 1006 of FIG. 1, operatively coupled to the repositionable structure to control positions and movement of the parts of the repositionable structure and instrument, as well as modify and/or otherwise provide a view to the user including, at various times, a view of the actual instrument shaft and/or a virtual instrument shaft representative of an equivalent position.

At block 502, the controller associated with a repositionable structure (e.g., the manipulators 140) may generate image data representative of a shaft and end (e.g., end effector) of an instrument (e.g., instrument 150) supported by the manipulators. In some embodiments, the controller receives and/or stores image data representative of the instrument from one or more image sensors (e.g., sensor system 1040) associated with the system (e.g., system 100). In further embodiments, the controller receives and/or stores image data representative of the instrument shaft and end from one or more other computing devices (e.g., the other computing device is communicatively coupled to the controller).

At block 505, the flow proceeds to either block 510 or 520 based on whether the system (e.g., the controller of the system) operates according to a first or second operating condition. Depending on the embodiment, the first operating condition may be an actual display condition, and the second operating condition may be a virtual display condition, as described with regard to FIGS. 4A and 4B above. As such, the first operating condition may indicate to the system to display a stream of image data (e.g., current video data) of the actual instrument shaft and/or manipulator, and the second operating condition may indicate to the system to display a modified stream of image data in which the actual instrument shaft and/or manipulator (or, in some embodiments, at least part of the actual instrument shaft and/or manipulator) is obscured, as described in more detail below.

If the controller operates according to the first operating condition, then flow proceeds to block 510. At block 510, the controller configures a display device (e.g., display device 1017) to display an actual representation of the instrument shaft based on the image data. At block 512, the controller controls motion of the instrument shaft based on control of a user input system relative to an actual position of the instrument shaft. In particular, the controller controls motion of the instrument shaft by receiving control signals for controlling the repositionable structure (e.g., from the user input system) and causing the repositionable structure to move relative to an actual position of the instrument shaft based on the control signals. As such, the controller controls motion of the instrument shaft by processing the received control signals and causing the repositionable structure and/or instrument shaft to move accordingly.

If the controller operates according to the second operating condition instead, then flow proceeds to block 520. At block 520, the controller modifies the image data to include a virtual representation of the instrument shaft and obscure the actual representation of the instrument shaft. In some embodiments, the image data is or includes a stream of image data and the controller modifies the stream of image data continuously (e.g., using updated image data in the stream of image data from an image sensor). In further embodiments, the controller generates the virtual representation of the instrument shaft responsive to detecting one or more changes associated between the image data and the updated image data.

Depending on the embodiment, the controller may generate a pose of the virtual representation. In some such embodiments, the controller generates the pose based on a metric indicative of case of an operator to utilize the user input system to advance the instrument shaft along a predicted trajectory. For example, if the controller generates the pose based on whether a pose is ergonomic and/or comfortable for a user to maintain and/or reach when advancing the instrument shaft along the predicted trajectory. Depending on the embodiment, the controller may determine whether a pose is ergonomic based on a range of motion or predicted range of motion for the user. In further embodiments, the metric is indicative of and/or otherwise based on a difference between an effective neutral pose of the virtual representation of the instrument shaft and an actual neutral pose of the instrument. For example, the neutral pose of the instrument may be representative of a position for the instrument shaft where the instrument end effector is set neutral relative to the instrument shaft (e.g., the wrist is positioned at a center position). As such, the controller may determine the metric based on a deviation from the neutral pose (e.g., by the instrument shaft and/or instrument wrist positioning). In still further embodiments, the controller may determine a range of the virtual representation of the instrument shaft by using the difference from the actual neutral pose. For example, if the virtual representation of the instrument shaft is displaying a neutral pose and the positioning differs from the actual neutral pose by a 35 degree angle, the controller may determine the metric either using the 35 degree difference as the new zero (e.g., “neutral pose” of the virtual instrument shaft representation), for example to 90 degrees, or using the original neutral pose, for example, limiting the range to −125 degrees to 55 degrees. Moreover, in still further embodiments, the range of motion is based on one or more motion limits of the instrument end effector and/or instrument wrist coupling the end effector to the shaft.

In further embodiments, the controller generates the pose based on a metric indicative of a pose of the actual representation of the instrument shaft relative to a target along a trajectory of the instrument shaft. In still further embodiments, the controller generates the pose based on a position of the target within the image data. In yet still further embodiments, the controller generates the pose of the virtual representation based on a determined preferred angle of approach to the target (e.g., of the user, of the instrument and instrument shaft, etc.).

In further embodiments, the controller generates the pose by determining a workplace volume for the user representative of one or more user motion limits. In some such embodiments, the controller may determine whether the clinical workspace needed for a procedure or step is within the actual workplace volume of the instrument (e.g., via image recognition techniques, by prompting a user to confirm details, by performing a calibration procedure, and/or any other such technique). The controller may then calculate a center of the workplace volume (e.g., a position-based center, an ergonomic center (e.g., a center neutral position relative to a user hand position), etc.) and/or a range of motion for the instrument using a view provided by one or more image sensors associated with the instrument (e.g., an endoscope view). The controller may then generate the pose relative to the ergonomic center (e.g., tilt clockwise by 30 degrees, move 5 units along an x-axis from the neutral position, etc.). In still further embodiments, the controller generates the pose based on a preferred pose associated with the user device. For example, the user device may have a preferred neutral pose stored in memory and/or otherwise input into the device. As such, the controller may generate the pose using the preferred pose as a baseline and/or default neutral pose (e.g., depending on a view (e.g., endoscope view)). In yet still further embodiments, the controller may receive one or more user preferences (e.g., via the user input system, from a database, from a communicatively coupled device, during a calibration process, etc.) and may generate the pose based on the one or more user preferences. Depending on the embodiment, the controller may generate the pose based on any combination of the above criteria, individually or in combination.

In some embodiments, the image sensor is a first image sensor and the controller obscures the actual representation of the instrument shaft by overlaying image data from a second image sensor over the actual representation. In further embodiments, the controller obscures the actual representation by synthetically generating a portion of the image data over the actual representation using an in-painting model. In still further embodiments, the image sensor is a stereoscopic image sensor and the controller obscures the actual representation of the instrument shaft based on image depth data from the stereoscopic image sensor.

At block 522, the controller configures the display device to display the modified image data. In some embodiments, the display device is a first display device and the system includes a second display device configured to display imaging data. Depending on the embodiment, the display device may be configured to display imaging data to an operator of the manipulator and/or instrument shaft, and the second display device may be configured to display imaging data to an observer of a procedure performed by the system. In further embodiments, the functionality of the display devices is swapped.

In some embodiments, the controller is further configured to display the modified image data on the second display device. For example, the controller may display the virtual representation to the observer via the second display device in addition to the operator via the first display device. In further embodiments, the controller is further configured to display the image data on the second display device. For example, the controller may display the actual representation to the observer via the second display device while displaying the virtual representation to the operator.

In still further embodiments, the controller is further configured to display a second virtual representation of the instrument shaft via the second display while displaying the first virtual representation of the instrument shaft via the first display. For example, the second virtual representation may include an annotated view of the instrument shaft, may be based on a point of view associated with the observer (e.g., displaying the virtual representation from the observer point of view rather than the operator point of view), may include a view representative of a hand position associated with the user (e.g., displaying the operator's hand position(s) virtually to display to an observer for learning purposes, monitoring purposes, recording purposes, etc.). In further embodiments, the first device may additionally or alternatively display the second virtual representation of the instrument shaft.

In further embodiments, the second display device may be multiple display devices (e.g., multiple monitors, computing devices, mobile devices, etc.). Similarly, the system may include multiple additional devices (e.g., a third display device, etc.) to display additional alternative view in line with those described herein. In still further embodiments, the system may include a single display device including multiple displays, each displaying a different view as described herein.

At block 524, the controller controls motion of the instrument shaft based on control of a user input system relative to the virtual representation of the instrument shaft. In particular, the controller receives control signals based on the virtual representation of the instrument shaft (e.g., for controlling the repositionable structure) from the user input system. The controller then processes the control signals to determine a desired motion relative to the actual position of the instrument shaft and/or repositionable structure to generate one or more modified control signals. The controller then causes the repositionable structure to move relative to the actual position of the instrument shaft based on the modified control signals (e.g., in accordance with the desired motion). Depending on the embodiment, the controller may determine the desired motion based on a predicted desired location for the instrument shaft and/or instrument end effector responsive to the control signals. For example, the virtual representation may show the instrument shaft positioned at a different angle than the actual position (e.g., to improve the ergonomic positioning of the operator's hands). The control signals may indicate that a user is trying to turn the instrument shaft, and the controller may predict (or receive a prediction regarding) a final location and/or position, and the controller may determine one or more modified control signals to position the instrument shaft and/or the repositionable structure accordingly. In further embodiments, the controller may determine that instrument shaft should follow the path, and the modified control signals reflect the same movement as the received control signals.

In some embodiments, the controller switches from operating according to the first operating condition to operating according to the second operating condition responsive to one or more indications. For example, the controller may switch operations responsive to a signal from the user to allow the user to switch modes of operation, responsive to a determination based on the metric indicative of case of an operator to utilize the user input system to advance the instrument shaft along a predicted trajectory, responsive to a determination based on the metric indicative of the pose of the actual representation of the instrument shaft relative to the target, etc.

In some embodiments, the controller is further configured to detect when the user is approaching a limit to a range of motion associated with the user and, in response, provide a warning to the user. For example, if the user surpasses a threshold distance from a neutral position associated with the instrument shaft, the controller may determine that the user is approaching the limit to the range of motion. Depending on the embodiment, the controller may be configured to be customizable for a particular user. For example, the controller may prompt a user on startup or on registration to perform one or more calibrations to determine a range of motion for the user. Similarly, the controller may prompt a user to provide and/or otherwise receive an indication of a range in which the user feels comfortable and may determine a threshold distance based on such. In further embodiments, the controller additionally detects how long a position has been held and provides a warning to the user further based on the period of time (e.g., if a threshold time period has been exceeded).

One or more components of the embodiments discussed in this disclosure, such as control system 1006, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

What is claimed:

1. A computer-assisted system for assisting a user in controlling an instrument including an instrument working portion coupled to an instrument shaft via an instrument wrist, the computer-assisted system comprising:
a repositionable structure configured to support (i) the instrument working portion via the instrument shaft and (ii) an image sensor configured to generate image data representative of the instrument working portion and the instrument shaft;
a user input system;
an output system comprising a display device; and
a control system operably coupled to the repositionable structure, the user input system, and the output system, the control system configured to:
while operating according to a first operating condition, (i) configure the display device to display an actual representation of the instrument shaft based on the image data and (ii) control motion of the instrument working portion and the instrument shaft by:
receiving one or more first control signals for controlling the repositionable structure from the user input system, and
causing the repositionable structure to move relative to an actual position of the instrument shaft based on the one or more first control signals;
while operating according to a second operating condition, modify the image data to include a virtual representation of the instrument shaft and obscure the actual representation of the instrument shaft; and
while operating according to the second operating condition, (i) configure the display device to display the modified image data and (ii) control motion of the instrument working portion and the instrument shaft by:
receiving one or more second control signals for controlling the repositionable structure from the user input system,
processing the one or more second control signals to determine a desired motion relative to the actual position to generate one or more modified second control signals, and
causing the repositionable structure to move relative to the actual position of the instrument shaft based on the one or more modified second control signals.

2. The computer-assisted system of claim 1, wherein, to modify the image data, the control system is further configured to:
while operating according to the second operating condition, receive a stream of image data from the image sensor;
modify the stream of image data to include the virtual representation of the instrument shaft; and
obscure the actual representation of the instrument shaft.

3. The computer-assisted system of claim 1, wherein the control system is configured to:
generate a pose of the virtual representation.

4. The computer-assisted system of claim 3, wherein the pose of the virtual representation is based on a metric indicative of ease of an operator to utilize the user input system to advance the instrument shaft along a predicted trajectory.

5. The computer-assisted system of claim 1, wherein the control system is further configured to:
switch from operating according to the first operating condition to operating according to the second operating condition responsive to a signal from the user to allow the user to switch modes of operation.

6. The computer-assisted system of claim 1, wherein the display device is a first display device configured to display imaging data to an operator of the repositionable structure, and the computer-assisted system further includes:
a second display device configured to display imaging data to an observer of a procedure performed by the repositionable structure.

7. The computer-assisted system of claim 1, wherein the virtual representation is a first virtual representation, the instrument is a first instrument, the instrument shaft is a first instrument shaft, and the control system is further configured to:
display a second virtual representation of a second instrument shaft on the display device while operating according to the second operating condition.

8. The computer-assisted system of claim 1, wherein the control system is further configured to:
detect when the user is approaching a limit to a range of motion associated with the user; and
in response to the detection, provide a warning to the user.

9. The computer-assisted system of claim 1, wherein the image sensor is a first image sensor, and, to modify the image data, the control system is configured to:
obscure the actual representation of the instrument shaft by overlaying image data generated by a second image sensor over the actual representation.

10. The computer-assisted system of claim 1, wherein, to modify the image data, the control system is configured to:
obscure the actual representation of the instrument shaft by generating, using an in-painting model, image data to overlay over the actual representation.

11. A computer-implemented method for assisting a user in controlling an instrument including an instrument working portion coupled to an instrument shaft via an instrument wrist, the computer-implemented method comprising:
while operating according to a first operating condition:
configuring, by one or more processors of a control system operably coupled to a repositionable structure configured to support (i) the instrument working portion via the instrument shaft and (ii) an image sensor configured to generate image data representative of the instrument working portion and the instrument shaft, a display device of an output system to display an actual representation of the instrument shaft based on the image data; and
controlling, by the one or more processors, motion of the instrument working portion and the instrument shaft by:
receiving, by the one or more processors, one or more first control signals for controlling the repositionable structure from a user input system, and
causing, by the one or more processors, the repositionable structure to move relative to an actual position of the instrument shaft based on the one or more first control signals; and
while operating according to a second operating condition:
modifying, by the one or more processors, the image data to include a virtual representation of the instrument shaft and obscure the actual representation of the instrument shaft;
configuring, by the one or more processors, the display device to display the modified image data; and
controlling, by the one or more processors, motion of the instrument working portion and the instrument shaft by:
receiving, by the one or more processors, one or more second control signals based on the virtual representation of the instrument and for controlling the repositionable structure from the user input system,
processing, by the one or more processors, the one or more second control signals to determine a desired motion relative to the actual position to generate one or more modified second control signals, and
causing, by the one or more processors, the repositionable structure to move relative to the actual position of the instrument shaft based on the one or more modified second control signals.

12. The computer-implemented method of claim 11, wherein modifying the image data comprises:
while operating according to the second operating condition, receiving, by the one or more processors, a stream of image data from the image sensor;
modifying, by the one or more processors, the stream of image data to include the virtual representation of the instrument shaft; and
obscuring, by the one or more processors, the actual representation of the instrument shaft.

13. The computer-implemented method of claim 11, further comprising:
generating, by the one or more processors, a pose of the virtual representation.

14. The computer-implemented method of claim 13, wherein the pose of the virtual representation is based on a metric indicative of ease of an operator to utilize the user input system to advance the instrument shaft along a predicted trajectory.

15. The computer-implemented method of claim 11, further comprising:
switching, by the one or more processors, from operating according to the first operating condition to operating according to the second operating condition responsive to a signal from the user to allow the user to switch modes of operation.

16. The computer-implemented method of claim 11, wherein the display device is a first display device configured to display imaging data to an operator of the repositionable structure, and the computer-implemented method further comprises:
causing, by the one or more processors, a second display device to display imaging data to an observer of a procedure performed by the repositionable structure.

17. The computer-implemented method of claim 11, wherein the virtual representation is a first virtual representation, the instrument is a first instrument, the instrument shaft is a first instrument shaft and the computer-implemented method further comprises:
displaying, by the one or more processors, a second virtual representation of a second instrument shaft on the display device while operating according to the second operating condition.

18. The computer-implemented method of claim 11, wherein the computer-implemented method further comprises:
detecting, by the one or more processors, when the user is approaching a limit to a range of motion associated with the user and, in response, provide a warning to the user.

19. The computer-implemented method of claim 11, wherein the image sensor is a first image sensor, and modifying the image data comprises:
obscuring, by the one or more processors, the actual representation of the instrument shaft by overlaying image data generated by a second image sensor over the actual representation.

20. A non-tangible computer-readable medium storing instructions for assisting a user in controlling an instrument including an instrument working portion coupled to an instrument shaft via an instrument wrist, wherein the instructions, when executed, cause a control system operably coupled to a repositionable structure configured to support (i) the instrument working portion via the instrument shaft and (ii) an image sensor configured to generate image data representative of the instrument working portion and the instrument shaft to:
while operating according to a first operating condition:
configure a display device of an output system to display an actual representation of the instrument shaft based on the image data; and
control motion of the instrument working portion and the instrument shaft by:
receive one or more first control signals for controlling the repositionable structure from a user input system, and
cause the repositionable structure to move relative to an actual position of the instrument shaft based on the one or more first control signals; and
while operating according to a second operating condition:
modify the image data to include a virtual representation of the instrument shaft and obscure the actual representation of the instrument shaft;
configure the display device to display the modified image data; and
control motion of the instrument working portion and the instrument shaft by:
receiving one or more second control signals based on the virtual representation of the instrument and for controlling the repositionable structure from the user input system,
processing the one or more second control signals to determine a desired motion relative to the actual position to generate one or more modified second control signals, and
causing the repositionable structure to move relative to the actual position of the instrument shaft based on the one or more modified second control signals.
